(19) United States
(12) Patent Application Publication
(10) Pub. No.: US 2010/0086937 A1
(43) Pub. Date: Apr. 8, 2010

(54) METHOD TO DETECT TREPONEMA PALLIDUM IMMUNOLOGICAL MARKERS FOR THE DIAGNOSIS OF SYPHILIS

(75) Inventors: Zhong Chen, Sandy, UT (US); Ning Liu, Beijing (CN); Yancun Li, Beijing (CN)

Correspondence Address: WEILI CHENG, CLAYTON, HOWARTH & CANNON, P.C., P.O.BOX 1909, SANDY, UT, 84091, US

(73) Assignee: CMED TECHNOLOGIES LTD., Road Town, Tortola (VG)

(21) Appl. No.: 12/442,623

(22) PCT Filed: Sep. 7, 2007
(86) PCT No.: PCT/US07/77867

§ 371 Date: Mar. 24, 2009

Related U.S. Application Data
(60) Provisional application No. 60/827,158, filed on Sep. 27, 2006.

(51) Int. Cl. G01N 33/53

(52) U.S. Cl. 435/7.1

(57) ABSTRACT

This invention discloses using SPR technology to qualitatively detect the presence of Treponema pallidum immunological markers in a serum sample, which can be used for the diagnosis of syphilis. It also discloses an efficient formula to make a mixed SAM that can greatly enhance the immobilization ability of the metal surface in SPR based techniques, which is good for the immobilization of representative antigens used to detect the respective Treponema pallidum immunological markers (antibodies) in blood for the diagnosis of syphilis.

### CROSS-REFERENCE TO RELATED APPLICATIONS

This invention claims priority, under 35 U.S.C. § 120, to the United States Provisional Patent Application No. 60/827,158 filed on 27 Sep. 2006, which is incorporated by reference herein.

### TECHNICAL FIELD

The present invention relates to a method of using SPR technology to detect the presence of different Treponema pallidum immunological markers in blood.

### INDUSTRIAL APPLICABILITY

It has been recognized that it would be advantageous to develop a label-free and high-throughput technique to simultaneously detect the presence of different Treponema pallidum immunological markers in blood. The METHOD TO DETECT TREPONEMA PALLIDUM IMMUNOLOGICAL MARKERS FOR THE DIAGONOSIS OF SYPHILIS relates to a novel method of using SPR technology to qualitatively detect Treponema pallidum immunological markers, which can be used for the diagnosis of syphilis. The METHOD TO DETECT TREPONEMA PALLIDUM IMMUNOLOGICAL MARKERS FOR THE DIAGONOSIS OF SYPHILIS provides an efficient formula to make a mixed SAM in and a method of using thereof for the immobilization of relevant antigen proteins in an SPR system for the detection of Treponema pallidum immunological markers in blood.

### DISCLOSURE OF THE INVENTION

Surface plasmon resonance (SPR) technology has been employed for quantitative and qualitative analysis in analytical chemistry, biochemistry, physics and engineering. SPR technology has become a leading technology in the field of direct real-time observation of biomolecular interactions.

SPR technology is highly sensitive to changes that occur at the interface between a metal and a dielectric medium (e.g., water, air, etc). In general, a high-throughput SPR instrument consists of an auto-sampling robot, a high resolution CCD (charge-coupled device) camera, and gold or silver-coated glass slide chips each with more than 4 array cells embedded in a plastic support platform.

SPR technology exploits surface plasmons (special electromagnetic waves) that can be excited at certain metal interfaces, most notably silver and gold. When incident light is coupled with the metal interface at angles greater than the critical angle, the reflected light exhibits a sharp attenuation (SPR minimum) in reflectivity owing to the resonant transfer of energy from the incident light to a surface plasmon. The incident angle (or wavelength) at which the resonance occurs is highly dependent upon the refractive index in the immediate vicinity of the metal surface. Binding of biomolecules at the surface changes the local refractive index and results in a shift of the SPR minimum. By monitoring changes in the SPR signal, it is possible to measure binding activities at the surface in real time. Traditional SPR spectroscopy sensors, which measure the entire SPR curve as a function of angle or wavelength, have been widely used, but offer limited throughput. The high-throughput capability of a high-throughput SPR instrument is largely due to its imaging system. The development of SPR imaging allows for the simultaneous measurement of thousands of biomolecule interactions.

Typically, a SPR imaging apparatus consists of a coherent p-polarized light source expanded with a beam expander and consequently reflected from a SPR active medium to a detector. A CCD camera collects the reflected light intensity in an image. SPR imaging measurements are performed at a fixed angle of incidence that falls within a linear region of the SPR dip; changes in light intensity are proportional to the changes in the refractive index caused by binding of biomolecules to the surface. As a result, gray-level intensity correlates with the amount of material bound to the sensing region. In addition, one of the factors determining the sensitivity of a SPR imaging system is the intensity of the light source. The signal strength from the metal surface is linearly proportional to the incoming light strength, so a laser light source is preferred over light-emitting diode and halogen lamps.

The SPR instrument is an optical biosensor that measures binding events of biomolecules at a metal surface by detecting changes in the local refractive index. The depth probed at the metal-aqueous interface is typically 200 nm, making SPR a surface-sensitive technique ideal for studying interactions between immobilized biomolecules and a solution-phase analyte. SPR technology offers several advantages over conventional techniques, such as fluorescence or ELISA (enzyme-linked immunosorbent assay) based approaches. First, because SPR measurements are based on refractive index changes, detection of an analyte is label free and direct. The analyte does not require any special characteristics or labels (radioactive or fluorescent) and can be detected directly, without the need for multistep detection protocols. Secondly, the measurements can be performed in real time, allowing the user to collect kinetic data, as well as thermodynamic data. Lastly, SPR is a versatile technique, capable of detecting analytes over a wide range of molecular weights and binding affinities. Therefore, SPR technology is a powerful tool for studying biomolecule interactions. So far, in research settings, SPR based techniques have been used to investigate protein-peptide interactions, cellular ligation, protein-DNA interactions, and DNA hybridization. However, SPR based approaches have not yet been explored in detecting Treponema pallidum immunological markers in blood for the diagnosis of syphilis.

Treponema pallidum is the microaerophilic spirochete that causes syphilis, a systemic venereal disease with multiple clinical presentations. Other closely related treponemas cause pinta (Treponema carateum), yaws (Treponema pallidum subspecies pertenue), and bejel (Treponema pallidum subspecies endemicum).

In general, the initial infection causes an ulcer at the site of infection; however, the bacteria move throughout the body, damaging many organs over time. Although treatment with penicillin in the early stages may be successful, the early symptoms of syphilis can be very mild, and many people do not seek treatment when they first become infected. This delay in seeking treatment is harmful because the damage to the organs in late syphilis cannot be reversed. Also of increasing concern is the risk of transmitting and acquiring the human immunodeficiency virus (HIV) that causes AIDS via open ulcers caused by syphilis.

Medical experts describe syphilis as having primary, secondary, latent, and tertiary (late) stages. An infected person who has not been treated may infect others during the first two stages, which usually last one to two years. The bacteria spread from the initial ulcer of an infected person to the skin or mucous membranes of the genital area, the mouth, or the anus of a sexual partner. The bacteria can also pass through broken skin on other parts of the body. In its late stages, untreated syphilis, although not contagious, can cause serious heart abnormalities, mental disorders, blindness, other neurologic problems, and even death.

The first symptom of primary syphilis is an ulcer called a chancre. The chancre can appear within 10 days to three months after exposure, but it generally appears within two to six weeks. The chancre is usually found on the part of the body exposed to the partner's ulcer, such as the penis, the vulva, or the vagina. A chancre also can develop on the cervix, tongue, lips, or other parts of the body. Because the chancre may be painless and may occur inside the body, it may go unnoticed. Although the chancre disappears within a few weeks whether or not a person is treated, if the infection is not treated during the primary stage, about one-third of those infected will progress to the chronic stages of syphilis.

Secondary syphilis is often marked by a skin rash that is characterized by brown sores about the size of a penny. The rash appears anywhere from three to six weeks after the chancre disappears. While the rash may cover the whole body, the palms of the hands and soles of the feet are the most common sites of presentation. Because active bacteria are present in these sores, any physical contact, sexual or nonsexual, with the broken skin of an infected person may spread the infection at this stage. The rash usually heals within several weeks or months. Other symptoms may also occur such as mild fever, fatigue, headache, sore throat, patchy hair loss, and swollen lymph glands throughout the body. These symptoms may be very mild and, like the chancre of primary syphilis, will disappear without treatment.

The signs of secondary syphilis may come and go over the next one to two years. If untreated, syphilis may lapse into a latent stage during which the disease is no longer contagious and no symptoms are present. Although many individuals who are not treated will suffer no further consequences of the disease, approximately one-third of those who have secondary syphilis develop the complications of late, or tertiary, syphilis.

In the tertiary stage of syphilis, bacteria damage the heart, eyes, brain, nervous system, bones, joints, or almost any other part of the body. This stage can last for years, or even decades. Late syphilis can result in mental illness, blindness, other neurologic problems, heart disease, and even death.

During the early stages of infection, syphilis bacteria also frequently invade the nervous system, and approximately three to seven percent of persons with untreated syphilis develop neurosyphilis. However, development of neurosyphilis can take up to twenty years and some persons with neurosyphilis never develop any symptoms. Those who do present symptoms may experience headaches, stiff necks, and fever, which result from an inflammation of the lining of the brain. Seizures and symptoms of stroke such as numbness, weakness, or visual problems may also afflict those patients with neuro syphilis. Although neurosyphilis can be treated, treatment may be more difficult and its course may be different in persons infected with HIV.

Syphilis usually is treated with penicillin, administered by injection. Other antibiotics are used for treating patients allergic to penicillin. A patient typically loses the ability to transmit syphilis within 24 hours from initiating therapy. Some infected individuals, however, do not respond to the usual doses of penicillin. Therefore, it is important that patients undergoing treatment for syphilis are monitored through periodic blood tests to ensure that the infectious agent has been completely destroyed. Persons with neurosyphilis may need to be re-tested for up to two years after treatment. In all stages of syphilis, proper treatment may cure the disease, but in late syphilis, damage already done to body organs cannot be reversed. Screening and treatment of infected individuals, or secondary prevention, is one of the few options available for preventing the advanced stages of syphilis disease. Testing and treatment early in pregnancy is the best way to prevent syphilis in infants and should be a routine part of prenatal care. A vital component in the successful treatment and prevention of syphilis is early and accurate detection of Treponema pallidum infection.

Due to the sometimes serious and life threatening effects of syphilis infection, and the risk of transmitting or contracting HIV, specific and early diagnosis of the infection is essential. Syphilis, however, has sometimes been called “the great imitator” because its early symptoms are similar to those of many other diseases. Therefore, a doctor usually does not rely upon recognition of the signs and symptoms of syphilis, but performs both microscopic identification of syphilis bacteria and blood tests.

To diagnose syphilis by a microscopic identification of the bacterium, the physician may take a scraping from the surface of the ulcer or chancre and examine it under a special “dark-field” microscope to detect the organism. However, dark-field microscopy requires considerable skill and is prone to misinterpretation. For these reasons, most cases of syphilis are diagnosed serologically, i.e. by the detection of antibodies to Treponema pallidum and/or cardiolipin. Specific IgM antibodies are detectable 14 days after infection and IgG antibodies appear about 4 weeks after infection. Only before that time direct detection of the causal agent from infected tissue of the primary lesion is another decisive diagnostic criterion. Serologic detection methods may be divided into three groups depending upon the nature of antibodies detected.

Nontreponemal tests: such as the cardiolipin microflocculation test (CMT), which is known in the English-speaking world as the Venereal Disease Research Laboratory Test (VDRL test), the rapid plasma reagin test (RPR test) and the cardiolipin complement binding reaction (cardiolipin CBR) are based on the detection of antibodies to cardiolipin. These tests reveal positive results 3-5 weeks after infection or approx. 7-10 days after appearance of the primary lesion. Sensitivity is 60 to 87% in the primary stage and may be as high as 100% in secondary syphilis. Sensitivity does, however, fall in the later stages of the disease, such that up to 30% of the late stages are no longer reactive. When the VDRL test is performed quantitatively, titer may be correlated to the activity of the disease. The disadvantage of this test is the large proportion of 0.3-0.9% of false positive test results when screening blood donors and the occurrence of false negative results in sera having an elevated titer due to the prozone phenomenon, which may be observed in 1-2% of cases in the VDRL test in secondary syphilis.

Treponema-specific tests: antibodies to the endoflagellae of Treponema pallidum are formed as syphilis progresses. As a result of antigen relatedness, these antibodies also react with the endoflagellae of other species of treponema. The endoflagellae of Treponema phagedenis (biotype Reiter) have thus also been used as an antigen for the diagnosis of syphilis. In the flagellum ELISA, the cut off for a positive test result is a compromise between sensitivity and specificity, as a result of which 0.8% of results are false positives and 2.7% false negatives.

Treponema pallidum-specific tests: these tests detect antibodies that react with Treponema pallidum or antigen preparations from this pathogen. These test systems include the Treponema pallidum haemagglutination test (TPHA), the fluorescent Treponema pallidum antibody absorption test (FTA-ABS) and the Nelson test (Treponema pallidum immobilisation test, TPI) together with ELISA systems based on sonicate antigen. TPHA and FTA-ABS are generally used in diagnostics.

At present, serological tests for detecting Treponema pallidum immunological markers are performed by using fluorescent or enzymic label based techniques that may be procedure-tedious and less accurate in quantification. In addition, fluorescent or enzymic label based techniques cannot detect all the immunological markers simultaneously. SPR technology has the ability of providing unlabeled, high-throughput, and on-line parallel analysis. The present invention demonstrates that SPR technology can be used as a powerful tool for the simultaneous detection of Treponema pallidum immunological markers, mainly including the antibodies to cardiolipin , the endoflagellae of Treponema pallidum , and Treponema pallidum or antigen preparations from this pathogen.

### REFERENCES

Mullett W M, Lai E P, Yeung J M. Surface plasmon resonance-based immunoassays. Methods. 2000 September;22(1):77-91.
Cao C, Kim J P, Kim B W, Chae H, Yoon H C, Yang S S, Sim S J. A strategy for sensitivity and specificity enhancements in prostate specific antigen-alpha1-antichymotrypsin detection based on surface plasmon resonance. Biosens Bioelectron. 2006 May 15;21(11):2106-13.
Choi S H, Lee J W, Sim S J. Enhanced performance of a surface plasmon resonance immunosensor for detecting Ab-GAD antibody based on the modified self-assembled monolayers. Biosens Bioelectron. 2005 Aug. 15;21(2):378-83.
Lee, J. W., Cho, S. M., Sim, S. J., Lee, J., 2005. Characterization of selfassembled monolayer of thiol on a gold surface and the fabrication of a biosensor chip based on surface plasmon resonance for detecting anti-GAD antibody. Biosens. Bioelectron. 20, 1422-1427.
Nedelkov D, Nelson R W. Surface plasmon resonance mass spectrometry: recent progress and outlooks. Trends Biotechnol. 2003 July;21(7):301-5. Review.
Flagellum ELISA, R. V. W. van Eijk et al., Genitourin. Med. 62, 367-372 (1988) Young et al., J. Clin. Pathol. 45, 37-41, (1992)
Ijsselmuiden et al., J. Clin. Microbiol. 27, 152-157, (1989)
Burdash et al., J. Clin. Microbiol. 25, 808-811, (1987)

### MODES FOR CARRYING OUT THE INVENTION

Before the present method of using SPR technology of label-free and high-throughput technique to simultaneously detect the presence of different Treponema pallidum immunological markers in blood is disclosed and described, it is to be understood that this invention is not limited to the particular configurations, process steps, and materials disclosed herein as such configurations, process steps, and materials may vary somewhat. It is also to be understood that the terminology employed herein is used for the purpose of describing particular embodiments only and is not intended to be limiting since the scope of the present invention will be limited only by the appended claims and equivalents thereof.

It must be noted that, as used in this specification and the appended claims, the singular forms “a,” “an,” and “the” include plural referents unless the context clearly dictates otherwise. Thus, for example, reference “an immunological marker” includes reference to two or more such markers.

In describing and claiming the present invention, the following terminology will be used in accordance with the definitions set out below.

“Proteins” and “peptides” are well-known terms in the art, and are not precisely defined in the art in terms of the number of amino acids that each includes. As used herein, these terms are given their ordinary meaning in the art. Generally, peptides are amino acid sequences of less than about 100 amino acids in length, but can include sequences of up to 300 amino acids. Proteins generally are considered to be molecules of at least 100 amino acids.

As used herein, a “metal binding tag” refers to a group of molecules that can become fastened to a metal that is coordinated by a chelate. Suitable groups of such molecules include amino acid sequences including, but not limited to, histidines and cysteines (“polyamino acid tags”). Metal binding tags include histidine tags, defined below.

“Signaling entity” means an entity that is capable of indicating its existence in a particular sample or at a particular location. Signaling entities of the invention can be those that are identifiable by the unaided human eye, those that may be invisible in isolation but may be detectable by the unaided human eye if in sufficient quantity (e.g., colloid particles), entities that absorb or emit electromagnetic radiation at a level or within a wavelength range such that they can be readily determined visibly (unaided or with a microscope including an electron microscope or the like), or spectroscopically, entities that can be determined electronically or electrochemically, such as redox-active molecules exhibiting a characteristic oxidation/reduction pattern upon exposure to appropriate activation energy (“electronic signaling entities”), or the like. Examples include dyes, pigments, electroactive molecules such as redox-active molecules, fluorescent moieties (including, by definition, phosphorescent moieties), up-regulating phosphors, chemiluminescent entities, electrochemiluminescent entities, or enzyme-linked signaling moieties including horse radish peroxidase and alkaline phosphatase.

“Precursors of signaling entities” are entities that by themselves may not have signaling capability but, upon chemical, electrochemical, electrical, magnetic, or physical interaction with another species, become signaling entities. An example includes a chromophore having the ability to emit radiation within a particular, detectable wavelength only upon chemical interaction with another molecule. Precursors of signaling entities are distinguishable from, but are included within the definition of, “signaling entities” as used herein.

As used herein, “fastened to or adapted to be fastened”, in the context of a species relative to another species or to a surface of an article, means that the species is chemically or biochemically linked via covalent attachment, attachment via specific biological binding (e.g., biotin/streptavidin), coordinative bonding such as chelate/metal binding, or the like. For example, “fastened” in this context includes multiple chemical linkages, multiple chemical/biological linkages, etc., including, but not limited to, a binding species such as a peptide synthesized on a polystyrene bead, a binding species specifically biologically coupled to an antibody which is bound to a protein such as protein A, which is covalently attached to a bead, a binding species that forms a part (via genetic engineering) of a molecule such as GST or Phage, which in turn is specifically biologically bound to a binding partner covalently fastened to a surface (e.g., glutathione in the case of GST), etc. As another example, a moiety covalently linked to a thiol is adapted to be fastened to a gold surface since thiols bind gold covalently. Similarly, a species carrying a metal binding tag is adapted to be fastened to a surface that carries a molecule covalently attached to the surface (such as thiol/gold binding) and which molecule also presents a chelate coordinating a metal. A species also is adapted to be fastened to a surface if that surface carries a particular nucleotide sequence, and the species includes a complementary nucleotide sequence.

“Covalently fastened” means fastened via nothing other than by one or more covalent bonds. E.g. a species that is covalently coupled, via EDC/NHS chemistry, to a carboxylate-presenting alkyl thiol which is in turn fastened to a gold surface, is covalently fastened to that surface.

“Specifically fastened (or bound)” or “adapted to be specifically fastened (or bound)” means a species is chemically or biochemically linked to another specimen or to a surface as described above with respect to the definition of “fastened to or adapted to be fastened”, but excluding all non-specific binding.

“Non-specific binding”, as used herein, is given its ordinary meaning in the field of biochemistry.

As used herein, a component that is “immobilized relative to” another component either is fastened to the other component or is indirectly fastened to the other component, e.g., by being fastened to a third component to which the other component also is fastened, or otherwise is translationally associated with the other component. For example, a signaling entity is immobilized with respect to a binding species if the signaling entity is fastened to the binding species, is fastened to a colloid particle to which the binding species is fastened, is fastened to a dendrimer or polymer to which the binding species is fastened, etc. A colloid particle is immobilized relative to another colloid particle if a species fastened to the surface of the first colloid particle attaches to an entity, and a species on the surface of the second colloid particle attaches to the same entity, where the entity can be a single entity, a complex entity of multiple species, a cell, another particle, etc.

The term “sample” refers to any medium suspected of containing an analyte, such as a binding partner, the presence or quantity of which is desirably determined. The sample can be a biological sample such as a cell, cell lysate, tissue, serum, blood or other fluid from a biological source, a biochemical sample such as products from a cDNA library, an environmental sample such as a soil extract, or any other medium, biological or non-biological, including synthetic material, that can advantageously be evaluated in accordance with the invention.

A “sample suspected of containing” a particular component means a sample with respect to which the content of the component is unknown. The sample may be unknown to contain the particular component, or may be known to contain the particular component but in an unknown quantity.

As used herein, a “metal binding tag” refers to a group of molecules that can become fastened to a metal that is coordinated by a chelate. Suitable groups of such molecules include amino acid sequences, typically from about 2 to about 10 amino acid residues. These include, but are not limited to, histidines and cysteines (“polyamino acid tags”). Such binding tags, when they include histidine, can be referred to as a “poly-histidine tract” or “histidine tag” or “HIS-tag”, and can be present at either the amino- or carboxy-terminus, or at any exposed region of a peptide or protein or nucleic acid. A poly-histidine tract of six to ten residues is preferred for use in the invention. The poly-histidine tract is also defined functionally as being the number of consecutive histidine residues added to a protein of interest which allows for the affinity purification of the resulting protein on a metal chelate column, or the identification of a protein terminus through interaction with another molecule (e.g. an antibody reactive with the HIS-tag).

A “moiety that can coordinate a metal”, as used herein, means any molecule that can occupy at least two coordination sites on a metal atom, such as a metal binding tag or a chelate.

“Affinity tag” is given its ordinary meaning in the art. Affinity tags include, for example, metal binding tags, GST (in GST/glutathione binding clip), and streptavidin (in biotin/streptavidin binding). At various locations herein specific affinity tags are described in connection with binding interactions. It is to be understood that the invention involves, in any embodiment employing an affinity tag, a series of individual embodiments each involving selection of any of the affinity tags described herein.

The term “self-assembled monolayer” (SAM) refers to a relatively ordered assembly of molecules spontaneously chemisorbed on a surface, in which the molecules are oriented approximately parallel to each other and roughly perpendicular to the surface. Each of the molecules includes a functional group that adheres to the surface, and a portion that interacts with neighboring molecules in the monolayer to form the relatively ordered array. See Laibinis. P. E.; Hickman. J.: Wrighton. M. S.: Whitesides, G. M. Science 245, 845 (1989). Bain. C.; Evall. J.: Whitesides. G. M. J. Am. Chem. Soc. 111, 7155-7164 (1989), Bain, C.; Whitesides, G. M. J. Am. Chem. Soc. 111, 7164-7175 (1989), each of which is incorporated herein by reference. The SAM can be made up completely of SAM-forming species that form close-packed SAMs at surfaces, or these species in combination with molecular wires or other species able to promote electronic communication through the SAM (including defect-promoting species able to participate in a SAM), or other species able to participate in a SAM, and any combination of these. Preferably, all of the species that participate in the SAM include a functionality that binds, optionally covalently, to the surface, such as a thiol which will bind covalently to a gold surface. A self-assembled monolayer on a surface, in accordance with the invention, can be comprised of a mixture of species (e.g. thiol species when gold is the surface) that can present (expose) essentially any chemical or biological functionality. For example, they can include tri-ethylene glycol-terminated species (e.g. tri-ethylene glycol-terminated thiols) to resist non-specific adsorption, and other species (e.g. thiols) terminating in a binding partner of an affinity tag, e.g. terminating in a chelate that can coordinate a metal such as nitrilotriacetic acid which, when in complex with nickel atoms, captures a metal binding tagged-species such as a histidine-tagged binding species.

“Molecular wires” as used herein, means wires that enhance the ability of a fluid encountering a SAM-coated electrode to communicate electrically with the electrode. This includes conductive molecules or, as mentioned above and exemplified more fully below, molecules that can cause defects in the SAM allowing communication with the electrode. A non-limiting list of additional molecular wires includes 2-mercaptopyridine, 2-mercaptobenzothiazole, dithiothreitol, 1,2-benzenedithiol, 1,2-benzenedimethanethiol, benzene-ethanethiol, and 2-mercaptoethylether. Conductivity of a monolayer can also be enhanced by the addition of molecules that promote conductivity in the plane of the electrode. Conducting SAMs can be composed of, but are not limited to: 1) poly (ethynylphenyl) chains terminated with a sulfur; 2) an alkyl thiol terminated with a benzene ring; 3) an alkyl thiol terminated with a DNA base; 4) any sulfur terminated species that packs poorly into a monolayer; 5) all of the above plus or minus alkyl thiol spacer molecules terminated with either ethylene glycol units or methyl groups to inhibit non specific adsorption. Thiols are described because of their affinity for gold in ready formation of a SAM. Other molecules can be substituted for thiols as known in the art from U.S. Pat. No. 5,620,820, and other references. Molecular wires typically, because of their bulk or other conformation, create defects in an otherwise relatively tightly-packed SAM to prevent the SAM from tightly sealing the surface against fluids to which it is exposed. The molecular wire causes disruption of the tightly-packed self-assembled structure, thereby defining defects that allow fluid to which the surface is exposed to communicate electrically with the surface. In this context, the fluid communicates electrically with the surface by contacting the surface or coming in close enough proximity to the surface that electronic communication via tunneling or the like can occur.

The term “biological binding” refers to the interaction between a corresponding pair of molecules that exhibit mutual affinity or binding capacity, typically specific or non-specific binding or interaction, including biochemical, physiological, and/or pharmaceutical interactions. Biological binding defines a type of interaction that occurs between pairs of molecules including proteins, nucleic acids, glycoproteins, carbohydrates, hormones and the like. Specific examples include antibody/antigen, antibody/hapten, enzyme/substrate, enzyme/inhibitor, enzyme/cofactor, binding protein/substrate, carrier protein/substrate, lectin/carbohydrate, receptor/hormone, receptor/effector, complementary strands of nucleic acid, protein/nucleic acid repressor/inducer, ligand/cell surface receptor, virus/ligand, etc.

The term “binding” or “bound” refers to the interaction between a corresponding pair of molecules that exhibit mutual affinity or binding capacity, typically specific or non-specific binding or interaction, including biochemical, physiological, and/or pharmaceutical interactions. Biological binding defines a type of interaction that occurs between pairs of molecules including proteins, nucleic acids, glycoproteins, carbohydrates, hormones and the like. Specific examples include antibody/antigen, anti body/hapten, enzyme/substrate, enzyme/inhibitor, enzyme/cofactor, binding protein/substrate, carrier protein/substrate, lectin/carbohydrate, receptor/hormone, receptor/effector, complementary strands of nucleic acid, protein/nucleic acid repressor/inducer, ligand/cell surface receptor, virus/ligand, etc.

The term “binding partner” refers to a molecule that can undergo binding with a particular molecule. Biological binding partners are examples. For example, Protein A is a binding partner of the biological molecule IgG, and vice versa.

The term “determining” refers to quantitative or qualitative analysis of a species via, for example, spectroscopy, ellipsometry, piezoelectric measurement, immunoassay, electrochemical measurement, and the like. “Determining” also means detecting or quantifying interaction between species, e.g. detection of binding between two species.

The term “self-assembled mixed monolayer” refers to a heterogeneous self-assembled monolayer, that is, one made up of a relatively ordered assembly of at least two different molecules.

“Synthetic molecule”, means a molecule that is not naturally occurring, rather, one synthesized under the direction of human or human-created or human-directed control.

The present invention generally relates to a method of SPR technology to detect Treponema pallidum immunological markers. More specifically, the present invention relates to using SPR technology to qualitatively detect Treponema pallidum immunological markers, which can be used for the diagnosis of syphilis. In addition, the present invention provides an efficient formula to make a mixed SAM that can greatly enhance the immobilization ability of the metal surface, which is desirable for the immobilization of relevant antigen proteins for detection.

For the diagnosis of syphilis, representative antigen proteins used to detect the respective Treponema pallidum immunological markers (antibodies) in a serum sample, suitable for the present invention, can be agents of cardiolipin, the endoflagellae of Treponema pallidum, Treponema pallidum or antigen preparations from this pathogen. To enhance the sensitivity and specificity of the SPR immunoassay, a link layer is attached onto the gold film on the surface of a glass chip which serves as a functional structure for further modification of the gold film surface. So far, several immobilization chemistries are suitable for the formation of the link layer, including alkanethiols, hydrogel, silanes, polymer films and polypeptides. Moreover, there are several methods to attach the link layer onto the thin gold surface, such as the Langmuir-Blodgett film method and the self-assembled monolayer (SAM) approach.

The following examples will enable those skilled in the art to more clearly understand how to practice the present invention. It is to be understood that, while the invention has been described in conjunction with the preferred specific embodiments thereof, that which follows is intended to illustrate and not limit the scope of the invention. Other aspects of the invention will be apparent to those skilled in the art to which the invention pertains.

### EXAMPLE 1

### Detection of Treponema Pallidum Immunological Markers in Blood for the Diagnosis of Syphilis

(A) Testing sample: serum (about 2 ml)
(B) Representative antigen proteins used to detect the respective Treponema pallidum immunological markers (antibodies) in a serum sample: agents of cardiolipin, the endoflagellae of Treponema pallidum, and Treponema pallidum or antigen preparations from this pathogen.

### (C) Procedure:

Step one: Formation of a linking layer on the surface of a gold-film glass chip:

1. Cleanliness of substrate

Metal substrates (copper, silver, aluminum or gold) were firstly cleaned with strong oxidizing chemicals (“piranha” solution-H₂SO₄:H₂O₂) or argon plasmas , then the surfaces of these substrates were washed with ultra pure water and degassed ethanol. After rinsing, the substrates were dried with pure N₂ gas stream.

2. Preparation of self-assembled monolayers (SAMs)

Single-component or mixed self-assembled monolayers (SAMs) of organosulfur compounds (thiols, disulfides, sulfides) on the clean metal substrate have been widely applied for chemical modification to develop chemical and biological sensor chips.

Preparing SAMs on metal substrates was achieved by immersion of a clean substrate into a dilute (˜1-10 mM) ethanolic solution of organosulfur compounds for 12-18 h at room temperature.

Monolayers comprising a well-defined mixture of molecular structures are called “mixed” SAMs. There are three methods for synthesizing mixed SAMs: (1) coadsorption from solutions containing mixtures of alkanethiols (HS(CH₂)ₙR+HS(CH₂)ₙR′), (2) adsorption of asymmetric dialkyl disulfides (R(CH₂)ₘS—S(CH₂)ₙR′), and (3) adsorption of asymmetric dialkylsulfides (R(CH₂)ₘS(CH₂)ₙR′), where n and m are the number of methylene units (range from 3 to 21) and R represents the end group of the alkyl chain (—CH₃, —OH, —COOH, NH₂) active for covalently binding ligands or biocompatible substance. Mixed SAMs are useful for decreasing the steric hindrance of interfacial reaction that, in turn, is useful for studying the properties and biology of cells.

3. Modifying SAMs

Methods for modifying SAMs after their formation are critical for the development of surfaces that present the large, complex ligands and molecules needed for biology and biochemistry. There are two important techniques for modifying SAMs:

(1) Direct reactions with exposed functional groups

Under appropriate reaction conditions, terminal functional groups (—OH, —COOH) exposed on the surface of a SAM immersed in a solution of ligands can react directly with the molecules present in solution. Many direct immobilization techniques have been adapted from methods for immobilizing DNA, polypeptides, and proteins on SAMs.

(2) Activation of surfaces for reactions

An operationally different approach to the functionalization of the surfaces of SAMs is to form a reactive intermediate, which is then coupled to a ligand. In this invention, we chose epoxy activation method to couple polysaccharide or a swellable organic polymer. In detail, 2-(2-Aminoethoxy) ethanol (AEE) was coupled to carboxyl-functionalized SAM using peptide coupling reagents (N-hydroxysuccinimide/N-Ethyl-N′-(3-dimethylaminopropyl)-carbodiimide (EDC/NHS)), and the terminal hydroxyl groups were further reacted with epichlorohydrin to produce epoxy-functionalized surfaces. These were subsequently reacted with hydroxyl moieties of polysaccharide or organic polymer. Subsequently, the polysaccharide chains were carboxylated through treatment with bromoacetic acid more than one time. The resultant material offered for further functionalization with biomolecules.

Rather than using single-component for preparing the SAM in conventional methods, “mixed” SAMs were used in the present invention, which provides various functional groups and branching structures to decrease the steric hindrance of interfacial reaction that, in turn, is useful for studying the biomolecular interaction analysis.

In addition, the facile surface plasmon resonance senses through specific biorecognizable gold substrates in combination with dextran using 2-(2-Aminoethoxy) ethanol (AEE) as a crosslinking agent, not gold nanoparticles as reported. As reported, dextran-treated surface was normally reacted with bromoacetic acid only one time. In our experiments, multiple bromoacetic acid reactions were employed in order to improve the carboxylated degree of dextran surface. Therefore, linking layer on the surface of a gold-film glass chip of the present invention significantly decreases the steric hindrance of interfacial reaction that, in turn, is useful for ligands immobilization.

Step two: Immobilization of relevant antigens on the surface of the linking layer:

A dextran coated sensor chip was used in this invention. The surface of the chip matrix was first activated by injection of a suitable activating agent (such as EDC/NHS or EDC/sulfo-NHS); afterwards the activating agent was washed out and the ligand solution (the representative antigens in 10 mM acetate buffer) was injected. After coupling, the remaining active groups in the matrix were deactivated by injection of a suitable agent (such as ehanolamine solution), then the non-covalently bound ligand was washed out by a high ionic strength medium.

For most covalent immobilization methods, electrostatic preconcentration of the ligand in the surface matrix was achieved with 10 mM acetate buffer at a suitable pH(range from 3.5 to 5.5). In our experiments, the representative antigens were prepared in 10 mM acetate buffer with suitable pH at concentrations of 10-100 μg/ml.

For instance, the surface of a sensor chip was activated by EDC/NHS. The ligands(representative antigens) in the 10 mM acetate buffer with suitable pH were spotted onto sensor chip using a microarray printing device. 1 M ethanolamine hydrochloride (pH 8.5) was used to deactivate excess reactive esters and to remove non-covalently bound ligand. Printed arrays were incubated in a humid atmosphere for 1 h and stored dry at 4° C. prior to use.

An important consideration for reproducibility is the ability to control the amount of representative antigens spotted on the matrix. Ideally, identical amount of antigens should be immobilized in the same area. Therefore, the use of reproducible amount of antigens is a critical step to ensure accurate results, especially in high-density array systems. Spotted technologies for reproducible delivery of microarrays of biological samples are preferred.

There are two ligand-coupling ways:

1). Direct coupling

Amine coupling introduces N-hydroxysuccinimide esters into the surface matrix by modification of the carboxymethyl groups with a mixture of N-hydroxysuccinimide (NHS) and N-ethyl-N′-(dimethylaminopropyl)-carbodiimide (EDC). These esters then react spontaneously with amines and other nucleophilic groups on the ligand to form covalent links. Amine coupling is the most generally applicable coupling chemistry, which is recommended as the first choice for most applications.

For most chemical coupling methods, preconcentration of a ligand on the surface matrix is important for efficient immobilization of macromolecules. This preconcentration can be accomplished by electrostatic attraction between negative charges on the surface matrix (carboxymethyl dextran) and positive charges on the ligand at pH values below the ligand pI, and allows efficient immobilization from relatively dilute ligand solutions. Electrostatic preconcentration is less significant for low molecular weight ligands.

Several important notes for the direct coupling are described as follows:

HBS-EP(pH 7.4) was first recommended. PBS(pH7.4) could be used as well.

The optimal pH for ligand immobilization is critically affected by the pH and ionic strength of the coupling buffer. The optimal condition for immobilization of relevant antigens was 10 mM acetate buffer at pH 5.0.

EDC/NHS(0.2 M N-ethyl-N′-(dimethylaminopropyl) carbodiimide /0.05 M N-hydroxysuccinimide) was injected to activate the surface .

The ligand solution was printed to the activated sensor chip surface.

1 Methanolamine hydrochloride(pH 8.5) was used to deactivate unreacted NHS-esters. The deactivation process also removed any remaining electrostatically bound ligand.

2) Indirect coupling

Most macromolecules contain many groups that can participate in the amine coupling reaction, and immobilization is usually easy. There are, however, situations where other coupling methods may be preferable:

Ligands where the active site includes particularly reactive amino or other nucleophilic groups may lose biological activity on immobilization

In certain situations, the multiplicity of amine coupling sites may be a disadvantage. The average number of attachment points for proteins to the matrix is normally low.

Several important notes for the indirect coupling are described as follows:

(1) HBS-EP(pH 7.4) was first recommended. PBS(pH7.4) could be used as well.

(2) NHS/EDC was injected to activate the sensor chip surface.

(3) 20 μg/ml of streptavidin in 10 mM acetate buffer at pH 5.0 was injected.

(4)1 Methanolamine hydrochloride (pH 8.5) was injected to deactivate excess reactive esters and to remove non-covalently bound streptavidin.

(5) 10 μg/ml of biotinylated protein in HBS-EP(pH 7.4) was injected.

Step three: Testing a sample:

1. Preparation of the serum sample to reduce unwanted binding

Unwanted binding may cause binding of analyte to non-specific sites on the surface, or binding of non-analyte molecules in the sample to the surface or the ligand. It is preferred to prepare the serum sample in order to obtain the best results.

One or more steps can be done for the serum preparation illustrated as follows:

(1) Inclusion of a surface-active agent, such as Surfactant P20 or Tween, in buffers and samples could help to reduce binding to non-specific sites, but could not guarantee that all binding would be biospecific.

(2) The use of physiological (0.15 M) salt concentrations could reduce non-specific electrostatic effects in most cases.

(3) Addition of zwitterions, such as taurine or betaine, could also help to reduce non-specific electrostatic adsorption.

(4) Addition of carboxymethyl dextran at approximate 1 mg/ml to the sample could reduce non-specific binding to the dextran matrix by competition effects.

(5) Addition of other monoclonal antibody at approximate 10 ug/l˜10 ug/ml to a sample could amplify the signal.

(6) The serum sample could be diluted 2-10 fold by using 1-10% of BSA, 5-50% of Bovine Calf Sera, 10-50% of mouse serum or 10-50% of rabbit serum.

2. Sample testing

To qualitatively detect the presence of Treponema pallidum immunological markers (antibodies) in a serum sample, relevant antigens of representative Treponema pallidum immunological markers were immobilized on the surface of the linking layer at predetermined concentrations, which allowed the antigens to react with various Treponema pallidum immunological markers (antibodies) in the serum. Subsequently, the antibody-agtigen reaction was detected with SPR system according to the standard operation procedure.

For comparison purposes, the same serum sample was checked for the same antibodies as detected with SPR technology by using an ELISA method. The presence of different Treponema pallidum immunological markers in a serum sample detected by SPR technology was consistent with those detected by ELISA methods.

In summary, as illustrated from the above detailed description and examples, the present invention demonstrates that the concentrations of Treponema pallidum immunological markers in a serum sample were positively related to the RU. In addition, the present invention also provides a more efficient formula to make the dextran coated sensor chip for improved immobilization of related antigens used for Treponema pallidum immunological markers assessment. The present invention demonstrates that SPR technology can be used to reliably detect representative antigens coated on the linking layer and the antibody-antigen reactions and the presence of different Treponema pallidum immunological markers in a serum sample measured by SPR system were consistent with those as detected with ELISA methods.

It is to be understood that the above-described embodiments are only illustrative of application of the principles of the present invention. Numerous modifications and alternative embodiments can be derived without departing from the spirit and scope of the present invention and the appended claims are intended to cover such modifications and arrangements. Thus, while the present invention has been shown in the drawings and fully described above with particularity and detail in connection with what is presently deemed to be the most practical and preferred embodiment(s) of the invention, it will be apparent to those of ordinary skill in the art that numerous modifications can be made without departing from the principles and concepts of the invention as set forth in the claims.

1. An improved SPR biosensor chip for the detection of the presence of Treponema pallidum immunological markers in blood for the diagnosis of syphilis, prepared by forming a linking layer on the surface of a metal film on a glass chip and immobilizing of one or more Treponema pallidum relevant antigens on the surface of the linking layer.

2. The improved SPR biosensor chip according to claim 1, wherein the linking layer is prepared by preparing a mixed SAM of long-chain alkanethiols which can bind with biomolecules through its suitable reactive groups on one side and react with said gold film through a gold-complexing thiol on the other side, modifying and activating the mixed SAMs.

3. The improved SPR biosensor chip according to claim 1, wherein said metal film is treated with dextran using 2-(2-Aminoethoxy) ethanol (AEE) as a crosslinking agent and multiple bromoacetic acid reactions.

4. The improved SPR biosensor chip according to claim 2, wherein said mixed SAMs is prepared by one of the following: (1) coadsorption from solutions containing mixtures of alkanethiols (HS(CH₂)ₙR+HS(CH₂)ₙR′), (2) adsorption of asymmetric dialkyl disulfides (R(CH₂)ₘS—S(CH₂)ₙR′), and (3) adsorption of asymmetric dialkylsulfides (R(CH₂)ₘS(CH₂)ₙR′), wherein n and m are the number of methylene units which is an integer from 3 to 21) and R represents the end group of the alkyl chain (—CH₃, —OH, —COOH, NH₂) active for covalently binding ligands or biocompatible substance.

5. The improved SPR biosensor chip according to claim 2, wherein said modifying and activating the mixed SAMs is accomplished by an epoxy activation method to couple a polysaccharide or a swellable organic polymer comprising coupling 2-(2-Aminoethoxy) ethanol (AEE) to carboxyl-functionalized SAM using peptide coupling reagents (N-hydroxysuccinimide/N-Ethyl-N′-(3-dimethylaminopropyl)-carbodiimide (EDC/NHS)), and reacting with epichlorohydrin to produce epoxy-functionalized surfaces, which subsequently being reacted with hydroxyl moieties of the polysaccharide or organic polymer, the resulting polysaccharide chains are subsequently being carboxylated through treatment with bromoacetic acid multiple times.

6. The improved SPR biosensor chip according to claim 1, wherein said Treponema pallidum relevant antigens are one or more members selected from the group consisting of Treponema pallidum cardiolipin components, Treponema pallidum endoflagellae components, Treponema pallidum components, and antigen preparations of Treponema pallidum.

7. The improved SPR biosensor chip according to claim 1, wherein said metal is copper, silver, aluminum or gold.

8. A method for simultaneously detecting the presence of Treponema pallidum immunological markers in blood for the diagnosis of syphilis, comprising the steps of:
1) preparing a surface plasmon resonance (SPR) system comprising:
a) an improved SPR biosensor chip according to claim 1,
b) a spectrophotometric means for receiving a first signal and a second signal from said surface, said second signal being received at a time after binding of said Treponema pallidum immunological markers (antibodies) and said Treponema pallidum relevant antigens on said surface; and
c) means for calculating and comparing properties of said first received signal and said second received signal to determine the presence of said Treponema pallidum immunological markers (antibodies);
2) contacting a serum sample to be tested with said biosensor surface and spectrophotometrically receiving said first signal and said second signal;
3) calculating and comparing said calculated differences to signals received to determine the presence Treponema pallidum immunological markers in blood for the diagnosis of syphilis.

9. The method according to claim 8, wherein the linking layer is prepared by preparing a mixed SAM of long-chain alkanethiols which can bind with biomolecules through its suitable reactive groups on one side and react with said gold film through a gold-complexing thiol on the other side, modifying and activating the mixed SAMs.

10. The method according to claim 8, wherein said metal film is treated with dextran using 2-(2-Aminoethoxy) ethanol (AEE) as a crosslinking agent and multiple bromoacetic acid reactions.

11. The method according to claim 9, wherein said mixed SAMs is prepared by one of the following: (1) coadsorption from solutions containing mixtures of alkanethiols (HS(CH₂)ₙR+HS(CH₂)ₙR′), (2) adsorption of asymmetric diallyl disulfides (R(CH₂)ₘS—S(CH₂)ₙR′), and (3) adsorption of asymmetric dialkylsulfides (R(CH₂)ₘS(CH₂)ₙR′), wherein n and m are the number of methylene units which is an integer from 3 to 21 and R represents the end group of the alkyl chain (—CH₃; —OH, —COOH, NH₂) active for covalently binding ligands or biocompatible substance.

12. The method according to claim 9, wherein said modifying and activating the mixed SAMs is accomplished by an epoxy activation method to couple a polysaccharide or a swellable organic polymer comprising coupling 2-(2-Aminoethoxy) ethanol (AEE) to carboxyl-functionalized SAM using peptide coupling reagents (N-hydroxysuccinimide/N-Ethyl-N′-(3-dimethylaminopropyl)-carbodiimide (EDC/NHS)), and reacting with epichlorohydrin to produce epoxy-functionalized surfaces, which subsequently being reacted with hydroxyl moieties of the polysaccharide or organic polymer, the resulting polysaccharide chains are subsequently being carboxylated through treatment with bromoacetic acid multiple times.

13. The method according to claim 8, wherein said Treponema pallidum relevant antigens are one or more members selected from the group consisting of Treponema pallidum cardiolipin components, Treponema pallidum endoflagellae components, Treponema pallidum components, and antigen preparations of Treponema pallidum.

14. The method according to claim 8, wherein said metal is copper, silver, aluminum or gold.
